(19) United States
(12) Patent Application Publication
(10) Pub. No.: US 2015/0104511 A1
(43) Pub. Date: Apr. 16, 2015

(54) Pharmaceutical Antiretroviral Combinations Comprising Lamivudine, Festinavir and Nevirapine

(71) Applicant: Cipla Limited, Mumbai, MH (IN)

(72) Inventors: Geena Malhotra, Mumbai (IN); Shrinivas Madhukar Purandare, Mumbai (IN)

(21) Appl. No.: 14/382,444

(22) PCT Filed: Mar. 5, 2013
(86) PCT No.: PCT/GB2013/000092

§ 371 Date: Sep. 2, 2014

(30) Foreign Application Priority Data
Mar. 5, 2012 (IN) 583/MUM/2012

(51) Int. Cl. A61K 9/24; A61K 31/551; A61K 31/513; A61K 31/7072

(52) U.S. Cl. CPC A61K 9/209; A61K 31/7072; A61K 31/551; A61K 31/513
USPC 424/465; 514/50; 424/489

(57) ABSTRACT

The present invention relates to a pharmaceutical antiretroviral composition comprising lamivudine, festinavir and nevirapine, to a process for preparing such a composition and to the use of such a composition for the treatment and/or prophylaxis of diseases caused by retroviruses, especially acquired immune deficiency syndrome or an HIV infection.

### CROSS-REFERENCE TO RELATED APPLICATIONS

This application is a filing under 35 U.S.C. 371 of International Application No. PCT/GB2013/000092 filed Mar. 5, 2013, entitled “Pharmaceutical Antiretroviral Combinations Comprising Lamivudine, Festinavir and Nevirapine,” which claims priority to Indian Patent Application No. 583MUM/2012 filed Mar. 5, 2012, which applications are incorporated by reference herein in their entirety.

### FIELD OF INVENTION

The present invention relates to a pharmaceutical antiretroviral composition comprising a combination of antiretroviral agents, the manufacturing process thereof and use of the said composition for the treatment or prophylaxis of diseases caused by retroviruses, especially acquired immune deficiency syndrome or an HIV infection.

### BACKGROUND

Demographically the second largest country in the world, India also has the third largest number of people living with HIV/AIDS. As per the provisional HIV estimate of 2008-09, by NACO (National AIDS Control Organization) there are an estimated 22.7 lakh people living with HIV/AIDS in India. The HIV prevalence rate in the country is 0.29 percent.

Acquired Immune Deficiency Syndrome (AIDS) causes a gradual breakdown of the body's immune system as well as progressive deterioration of the central and peripheral nervous systems. Since its initial recognition in the early 1980's, AIDS has spread rapidly and has now reached epidemic proportions within a relatively limited segment of the population. Intensive research has led to the discovery of the responsible agent, human T-lymphotropic retrovirus 111 (HTLV-111), now more commonly referred to as the human immunodeficiency virus or HIV.

Human immunodeficiency virus (HIV) is the etiological agent of Acquired Immune Deficiency Syndrome (AIDS) that has created a major health care problem not only in India but also globally.

HIV is a member of the class of viruses known as retroviruses. The retroviral genome is composed of RNA, which is converted to DNA by reverse transcription. This retroviral DNA is then stably integrated into a host cell's chromosome and, employing the replicative processes of the host cells, produces new retroviral particles and advances the infection to other cells. HIV appears to have a particular affinity for the human T-4 lymphocyte cell which plays a vital role in the body's immune system. HIV infection of these white blood cells depletes this white cell population. Eventually, the immune system is rendered inoperative and ineffective against various opportunistic diseases.

The current strategy for the treatment of HIV infection is Highly Active Antiretroviral Therapy (HAART). HAART normally consists of a combination of two or more reverse transcriptase (RT) inhibitors (RTIs) and protease inhibitors (PIs) taken together which targets different steps in the viral replication cycle.

Currently available antiretroviral drugs for the treatment of HIV include nucleoside reverse transcriptase inhibitors (NRTI) or approved single pill combinations: zidovudine or AZT (Retrovir®), didanosine or DDI (Videx®), stavudine or D4T (Zenith®), lamivudine or 3TC (Epivir®), zalcitabine or DDC (Hivid®), abacavir succinate (Ziagen®), tenofovir disoproxil fumarate salt (Viread®), emtricitabine (Emtriva®), Combivir® (contains 3TC and AZT), Trizivir® (contains abacavir, 3TC and AZT); non-nucleoside reverse transcriptase inhibitors (NNRTI): nevirapine (Viramune®), delavirdine (Rescriptor®) and efavirenz (Sustiva®), peptidomimetic protease inhibitors or approved formulations: saquinavir (Invirase®, Fortovase®), indinavir (Crixivan®), ritonavir (Norvir®), nelfinavir (Viracept®), amprenavir (Agenerase®), atazanavir (Reyataz®), fosamprenavir (Lexiva®), Kaletra® (contains lopinavir and ritonavir), one fusion inhibitor enfuvirtide (T-20, Fuzeon®), Truvada® (contains Tenofovir and Emtricitabine) and Atripla® (contains fixed-dose triple combination of tenofovir, emtricitabine and efavirenz).

The goal of HAART therapy is to maximize viral suppression thus limiting and reversing damage to the immune system, leading to decline in opportunistic infections. The durability of response depends on various factors such as viral, drug and patient related factors. However, the emergence of drug-resistant HIV-1 mutants often results in the failure of therapy.

Viral factors include the genetic barrier to resistance development, the capacity to remain latent and ongoing replication. Drug related factors include the potency, tolerability and convenience of a regimen and pharmacologic barriers to resistance as a function of concentrations achieved by these drugs. However, the most important patient related factor is adherence, but other factors such as toxicities, quality of life, and psychosocial issues also need to be addressed to ensure the success of therapy.

Adherence is critical for the success of HAART. Numerous studies have documented that a high level of adherence is needed to ensure maximal and durable suppression of the virus (Paterson D L. Et al. Adherence to protease inhibitor therapy and outcomes in patients with HIV infection Annals of Internal Medicine, 2000; 133:21-30).

Various factors influence adherence, one of which is the use of different drug combinations, which are difficult to adhere to because of different dosage forms for administering each antiretroviral drug separately, this is particularly important in case of elderly patients or it may also be due to other factors such as food restrictions, treatment costs, difficulties in accessing care, and unavailability of drugs in remote places.

Since eradication of HIV is unlikely with currently available HAART and since the evidence for structured treatment interruption seems disappointing (Jintanat A. et al. Swiss HIV Cohort Study. Failures of 1 week on, 1 week off antiretroviral therapies in a randomized trial AIDS, 2003; 17:F33-F37), HIV therapy needs to be life-long coupled with high levels of adherence to the therapy; this is a demanding task for HIV infected patients due to various reasons like low morale, social stigma, low immunity attributed to the disease. Further, studies have shown that adherence to prescribed drugs over long treatment periods is generally poor. Non-adherence to HAART can lead to rebound in viral replication and, in presence of suboptimal drug concentrations as well as rapid development of drug resistance. The development of drug resistance can be disastrous because of the complexity and cost associated with second line regimens and the potential for transmission of drug resistant virus in the community.

Thus, development of a fixed dose combination is a main step in simplifying the multi-drug combination therapy for improving patient adherence to the therapy since such non-adherence may contribute to the development of viral resistance and treatment failure. Further, the multi-drug combination therapy reduces the cost and also provides development of a fixed dose combination. Another advantage is that patients prefer taking one pill twice a day as compared to three pills twice a day. Convenience increases adherence, which ultimately leads to durable response in therapy.

Combination therapy, thus, reduces the daily doses to be taken by patients and simplifies dosing schedule thereby increasing patient compliance. Combination therapy also increases the drug efficacy. Use of combination therapy can yield an equivalent antiviral effect with reduced toxicity. Further, it may also reduce the risk of giving the wrong dose (high or low) of individual drugs since high doses can lead to development of serious adverse events, low doses can lead to suboptimal drug concentrations and development of drug resistance.

WO2007/026156 discloses a pharmaceutical composition of larnivudine, stavudine and nevirapine for inhibiting human immunodeficiency virus (HIV).

WO92/20344, WO98/18477, and WO99/55372 disclose combinations of lamivudine with other reverse transcriptase inhibitors, in particular zidovudine.

U.S. Pat. No. 6,486,183 relates to the field of antivirals and in particular to HIV reverse transcriptase inhibitors and provides novel compounds, pharmaceutical compositions comprising these compounds and methods for the inhibition of HIV employing them.

WO2004/087169 relates to an invention which provides for a pharmaceutical composition useful for the treatment or prophylaxis of viral infections comprising nevirapine and at least one antiviral active compound, wherein base is selected from the group consisting of thymine, cytosine, adenine, guanine, inosine, uracil, 5-ethyluracil and 2,6-diaminopurine, or a pharmaceutically acceptable salt or prodrug thereof, an example of such antiviral active compound being alovudine.

Further, to meet the patient acceptance levels, patient adherence and high value treatment consideration prospects, specifically, to combat such dreadful disease/syndrome, a single complete package of such medicaments have still remained out of reach of the patients at large.

Hence, there exists a need to formulate a suitable pharmaceutical antiretroviral composition in a single unit dosage form, for example comprising lamivudine, festinavir and nevirapine, which would be convenient for patient administration thereby achieving patient adherence and exhibiting desirable dissolution.

Further, in spite of all the available antiretroviral formulations and various methods suggested in prior art, there have been difficulties incorporating lamivudine, festinavir and nevirapine in a fixed dose combination to provide a once or twice a day formulation which is stable and suitable for administration.

### OBJECTS OF THE INVENTION

The object of the present invention is to provide a pharmaceutical antiretroviral composition suitable for oral administration, optionally comprising one or more pharmaceutically acceptable excipients.

Another object of the present invention is to provide a pharmaceutical antiretroviral composition, optionally comprising one or more pharmaceutically acceptable excipients, for once or twice a day administration.

Yet another object of the present invention is to provide a novel pharmaceutical antiretroviral composition with ease of manufacture.

Still another object of the present invention is to provide a pharmaceutical antiretroviral composition for use in the treatment or prophylaxis of diseases caused by retroviruses, especially acquired immune deficiency syndrome or an HIV infection.

### SUMMARY OF THE INVENTION

According to one aspect of the present invention there is provided a pharmaceutical antiretroviral composition comprising lamivudine, festinavir and nevirapine, said composition optionally comprising one or more pharmaceutically acceptable excipients.

According to another aspect of the present invention there is provided a pharmaceutical antiretroviral composition comprising lamivudine, festinavir and nevirapine, optionally comprising one or more pharmaceutically acceptable excipients, in a single unit dosage form.

In a preferred embodiment, the pharmaceutical antiretroviral composition of the present invention comprises lamivudine, festinavir and nevirapine as the sole active pharmaceutical ingredients (APIs).

According to another aspect of the present invention there is provided a process of manufacturing a pharmaceutical antiretroviral composition comprising lamivudine, festinavir and nevirapine optionally with one or more pharmaceutically acceptable excipients.

According to yet another aspect of the present invention there is provided a method for the treatment or prophylaxis of diseases caused by retroviruses, especially acquired immune deficiency syndrome or an HIV infection, which method comprises administering a pharmaceutical antiretroviral composition comprising lamivudine, festinavir and nevirapine.

### DETAILED DESCRIPTION

As discussed above, there is a need to develop and formulate a suitable pharmaceutical antiretroviral composition comprising once or twice a day formulation of lamivudine, festinavir and nevirapine which would, not only, be convenient for patient administration but would also maintain patient adherence for such a therapy.

The present invention thus provides a pharmaceutical antiretroviral composition comprising lamivudine, festinavir and nevirapine as a combined preparation, for simultaneous or separate administration, preferably for use in the treatment of an HIV infection.

It will be appreciated from the above, that the respective therapeutic agents of the combined preparation can be administered simultaneously, either in the same or different pharmaceutical formulations or separately. If there is separate administration, it will also be appreciated that the subsequently administered therapeutic agents should be administered to a patient within a time scale so as to achieve, or more particularly optimize, synergistic therapeutic effect of the combined preparation.

Thus, the present invention provides a pharmaceutical antiretroviral composition comprising lamivudine, festinavir and nevirapine for once or twice a day administration, said composition optionally comprising one or more pharmaceutically acceptable excipients.

Further, there is also provided a pharmaceutical antiretroviral composition comprising lamivudine, festinavir and extended release nevirapine for once or twice a day administration. According to this embodiment, the composition is formulated to deliver nevirapine over an extended period of time upon administration relative to an immediate release nevirapine composition.

Suitably, there is provided a pharmaceutical antiretroviral composition comprising lamivudine, festinavir and nevirapine for once or twice a day administration; however the frequency of administration may depend on certain factors such as severity of the disease condition, dose of the active agent as well as the patient related factors.

The terms “Lamivudine”, “Festinavir”, and “Nevirapine” and are used in broad sense to include not only, “Lamivudine”, “Festinavir”, and “Nevirapine” per se but also, their pharmaceutically acceptable salts, pharmaceutically acceptable solvates, pharmaceutically acceptable hydrates, pharmaceutically acceptable enantiomers, pharmaceutically acceptable esters, pharmaceutically acceptable derivatives, pharmaceutically acceptable polymorphs, pharmaceutically acceptable prodrugs, and pharmaceutically acceptable complexes.

Nevirapine, is chemically known as 11-cyclopropyl-5,11-dihydro-4-methyl-6H-dipyrido[3,2-b:2′,3′-e][1,4]diazepin-6-one, belongs to a category of non-nucleoside reverse transcriptase inhibitor (NNRTI) which is used to treat infection by HIV-I (human immunodeficiency virus, type 1). Nevirapine binds directly to reverse transcriptase (RT) and blocks the RNA-dependent and DNA-dependent DNA polymerase activities by causing a disruption of the enzyme's catalytic site. A suitable dosage range of nevirapine for use in a pharmaceutical antiretroviral composition of the present invention is from 50 to 400 mg, for example 50, 100, 200 or 400 mg.

Lamivudine (also known as 3TC) is a synthetic analogue, chemically known as (2R˜cis)-4-Amino-1-[2-(hydroxymethyl) 1,3-oxathiolan-5-yl]-2(1H)-pyrimidinone. Lamivudine has also been referred to as (−)-1-[(2R,5S)2-(Hydroxymethyl)-1,3-oxathiolan-5-yl]cystosine, (Hydroxymethyl)-1,3-oxathiolan-5-yl]cystosine. Lamivudine has proven antiviral activity against HIV and other viruses such as HBV. It has been found that Lamivudine exhibits unexpected advantages when used in combination with known inhibitors of HIV replication. A suitable dosage range of lamivudine for use in a pharmaceutical antiretroviral composition of the present invention is from 150 to 300 mg.

Festinavir (4′-Ed4T) (2′,3′-didehydro-3′-deoxy-4′-ethynylthymidine; OBP-601) is a stavudine (d4T) analog with a 4′-ethynyl substitution and exhibits 5-10-fold fold improved potency and a reduced in vitro toxicity, including less effects on mtDNA, as compared to stavudine (d4T). Further, the active metabolite 4′-Ed4T triphosphate has a longer in vitro intracellular retention than the triphosphates of zidovudine (AZT) and stavudine (d4T) and hence consequently 4′-Ed4T exhibits more persistent anti-HIV activity after drug removal than the other thymidine analogs. The prolonged intracellular half-life of 4′-Ed4T metabolites appears to be partly due to reduced catabolism by thymidine phosphorylase and due to limited cellular efflux. In addition, 4′-Ed4T had a lower cellular and mitochondrial toxicity than stavudine (d4T) since cumulative exposure to stavudine (d4T) has the potential to cause disfiguring, painful and life-threatening side-effects, such as lipodystrophy, peripheral neuropathy and lactic acidosis. The 4′-ethynyl group provides additional binding energy through its interaction with a hydrophobic pocket in the active site of RT (Reverse Transcriptase). This interaction increases the affinity of 4′-Ed4T-triphosphate to RT (Reverse Transcriptase) approximately 5-fold compared to d4T-triphosphate and at the same time reduces the interactions with mtDNA pol γ. A suitable dosage range of festinavir for use in a pharmaceutical antiretroviral composition of the present invention is from 100 to 600 mg.

According to a preferred embodiment, the present invention provides a pharmaceutical antiretroviral composition comprising lamivudine, festinavir and nevirapine in a single unit dosage form.

According to another preferred embodiment, the present invention provides a pharmaceutical antiretroviral composition comprising lamivudine, festinavir and nevirapine in a nanosize (i.e. sub-micron) form.

According to yet another preferred embodiment, the present invention provides a pharmaceutical antiretroviral composition comprising lamivudine, festinavir and extended release nevirapine, wherein nevirapine is incorporated/presented in an extended release system.

As used herein, “extended release nevirapine” means nevirapine formulated to provide a reduction in dosing frequency as compared to an immediate-release nevirapine formulation as well as to provide an in vitro and/or in vivo drug release profile of extended duration, in particular relative to the release profile of an immediate release nevirapine formulation.

Further, the term “extended-release”, as used herein, refers to the release of an active ingredient from a pharmaceutical antiretroviral composition, in which the active ingredient is released over an extended period of time and/or at a particular location and is taken to encompass sustained-release, controlled-release, modified-release, prolonged-release, delayed-release, and the like.

Suitable nevirapine-containing extended release formulations may include, but are not limited to dissolution controlled release system, diffusion controlled release system, dissolution and diffusion controlled release system, ion exchange resin-drug complex, pH dependent formulation and osmotic pressure controlled system and any other release systems known to person skilled in the art.

According to the above embodiment, the pharmaceutical antiretroviral composition of the present invention may comprise nevirapine in an extended release form, wherein nevirapine is formulated with at least one hydrophilic and/or hydrophobic polymer and/or water swellable polymer. In one embodiment, nevirapine may be coated with one or more hydrophilic and/or hydrophobic polymers. In an alternative embodiment, nevirapine may be blended with one or more hydrophilic and/or hydrophobic polymers. In another embodiment, nevirapine may be provided as API:polymer complex with suitable ratios of the API and the hydrophilic and/or hydrophobic polymer.

Hydrophilic polymers that may be used in the pharmaceutical antiretroviral composition are well known in the art and include pharmaceutically acceptable polymeric materials having a sufficient number and distribution of hydrophilic substituents (such as hydroxy and carboxy groups) to impart hydrophilic properties to the polymer as a whole. The amount of hydrophilic polymer in the composition depends on the particular polymer selected, on the active pharmaceutical agent and on the desired extended release profile.

Examples of suitable pharmaceutically acceptable hydrophilic polymers for use in the pharmaceutical antiretroviral compositions of the present invention include one or more of, but not limited to, hydroxypropylmethylcellulose (HPMC, also known as hypromellose), hydroxypropylcellulose (HPC), methylcellulose, carmellose (carboxymethylcellulose), hydroxyethylcellulose (HEC), hydroxymethylcellulose, methylcellulose, carboxymethylcellulose, sodium carboxymethylcellulose, carboxymethylcellulose calcium, xanthan gum, sodium alginate, ammonium alginate, polyethylene oxide, potassium alginate, calcium alginate, propylene glycol alginate, alginic acid, polyvinyl alcohol, povidone, carbomer, guar gum, locust bean gum, potassium pectate, potassium pectinate, polyvinylpyrrolidone, polysaccharide, polyalkylene oxides, polyalkyleneglycol, starch and derivatives and crosslinked homopolymers and copolymers of acrylic acid, or mixtures thereof.

According to one aspect of the present invention, the hydrophilic polymer is included in an amount of from about 5% to about 50%, preferably from about 10% to about 35%, by weight of the composition.

Examples of suitable pharmaceutically acceptable hydrophobic polymers for use in the pharmaceutical antiretroviral compositions of the present invention include one or more of, but not limited to, ethyl cellulose, cellulose acetate, cellulose acetate butyrate, cellulose acetate phthalate, cellulose acetate trimellitate, hydroxypropyl methylcellulose phthalate, poly (alkyl) methacrylate, and copolymers of acrylic or methacrylic acid esters, ammonio methyacrylate copolymer, methyacrylic acid copolymers, methacrylic acid-acrylic acid ethyl ester copolymer, methacrylic acid esters neutral copolymer, polyvinyl acetate, waxes, such as, beeswax, carnauba wax, microcrystalline wax, candelilla wax, spermaceti, montan wax, hydrogenated vegetable oil, lecithin, hydrogenated cottonseed oil, hydrogenated tallow, paraffin wax, shellac wax, petrolatum, ozokerite, and the like, as well as, synthetic waxes, e.g., polyethylene, and the like; fatty acids such as, stearic acid, palmitic acid, lauric acid, eleostearic acids, and the like; fatty alcohols, such as, lauryl alcohol, cetostearyl alcohol, stearyl alcohol, cetyl alcohol and myristyl alcohol; fatty acid esters, such as, glyceryl monostearate, glycerol monooleate, acetylated monoglycerides, tristearin, tripalmitin, cetyl esters wax, glyceryl palmitostearate and glyceryl behenate; vegetable oil, such as, hydrogenated castor oil; mineral oil or mixtures thereof.

According to one aspect of the present invention, the hydrophobic polymer is included in an amount of from about 5% to about 50%, preferably from about 10% to about 35%, by weight of the composition.

According to the present invention, pharmaceutically acceptable water-swellable polymer for use in the pharmaceutical antiretroviral composition of the present invention may comprise one or more, polyethylene oxide having a molecular weight of 100,000 to 8,000,000; poly (hydroxy alkyl methacrylate) having a molecular weight of from 30,000 to 5,000,000; poly (vinyl) alcohol, having a low acetal residue, which is cross-linked with glyoxal, formaldehyde or glutaraldehyde and having a degree of polymerization of from 200 to 30,000; a mixture of methyl cellulose, cross-linked agar and carboxymethyl cellulose; a water-insoluble, water-swellable copolymer produced by forming a dispersion of a finely divided copolymer of maleic anhydride with styrene, ethylene, propylene, butylene or isobutylene cross-linked with from 0.001 to 0.5 moles of saturated cross-linking agent per mole of maleic anhydride in the copolymer; Carbopol® carbomer which is as acidic carboxy polymer having a molecular weight of 450,000 to 4,000,000; Cyanamer® polyacrylamides; cross-linked water swellable indene-maleic anhydride polymers; Goodrich® polyacrylic acid having a molecular weight of 80,000 to 200,000; starch graft copolymers; Aqua Keeps® acrylate polymer polysaccharides composed of condensed glucose units such as diester cross-linked polyglucan, and the like; Amberlite® ion exchange resins; Explotab® sodium starch glycolate; Ac-Di-Sol® croscarmellose sodium or mixtures thereof.

As discussed above and hereinafter, in one embodiment the pharmaceutical antiretroviral composition of the present invention comprises lamivudine, festinavir along with one or more pharmaceutically acceptable excipients to form a layer, and nevirapine along with one or more extended release polymers and one or more pharmaceutically acceptable excipients to form another layer, which layers are blended and/or layered to provide a single unit dosage form.

Suitably, the pharmaceutical antiretroviral compositions according to the present invention are presented in dosage forms, conveniently in unit dosage form, and include dosage forms suitable for oral and buccal administration such as, but not limited to, tablets, capsules (filled with powders, pellets, beads, mini-tablets, pills, micro-pellets, small tablet units, MUPS, disintegrating tablets, dispersible tablets, granules, and microspheres, multiparticulates), soft gelatin capsules, sachets (filled with powders, pellets, beads, mini-tablets, pills, micro-pellets, small tablet units, MUPS, disintegrating tablets, dispersible tablets, granules, and microspheres, multiparticulates), sprinkles, liquid dosage forms (liquids, liquid dispersions, suspensions, solutions, emulsions, sprays, syrup, spot-on and the like), injection preparations, gels, aerosols, ointments, creams, controlled release formulations, lyophilized formulations, delayed release formulations, extended release formulations, pulsatile release formulations, dual release formulations etc. and the like, are also included in the scope of this invention.

In another aspect, the present invention provides a pharmaceutical antiretroviral composition comprising lamivudine, festinavir and nevirapine in a kit form.

According to one embodiment the pharmaceutical antiretroviral composition in a kit form may contain a separate unit dosage form comprising lamivudine and nevirapine and a separate unit dosage form comprising festinavir.

According to another embodiment the pharmaceutical antiretroviral composition in a kit form may contain a separate unit dosage form comprising lamivudine and festinavir and a separate unit dosage form comprising nevirapine.

According to yet another embodiment pharmaceutical antiretroviral composition in a kit form may contain a separate unit dosage form comprising nevirapine and festinavir and a separate unit dosage form comprising lamivudine.

According to a further embodiment the pharmaceutical antiretroviral composition in a kit form may contain a separate unit dosage form comprising nevirapine, a separate unit dosage form comprising lamivudine and a separate unit dosage form comprising festinavir.

It is further well known in the art that a tablet formulation is the preferred solid dosage form due to its greater stability, less risk of chemical interaction between different medicaments, smaller bulk, accurate dosage, and ease of production.

Solid unit dosage forms, according to the present invention, include conventional dosage forms such as capsules (filled with powders, pellets, beads, mini-tablets, pills, micro-pellets, small tablet units, MUPS, disintegrating tablets, dispersible tablets, granules, and microspheres, multiparticulates and the like), soft gelatin capsules, sachets (filled with powders, pellets, beads, mini-tablets, pills, micro-pellets, small tablet units, MUPS, disintegrating tablets, dispersible tablets, granules, and microspheres, multiparticulates) and sprinkles and the like are included within the scope of this invention.

Preferably, the solid unit dosage forms, according to the present invention are in the form of tablets.

According to a preferred embodiment, the pharmaceutical antiretroviral composition may be a single unit dosage form wherein the APIs and excipients are present in a single layer entity (such as a tablet or tablet in a capsule).

According to another preferred embodiment, the pharmaceutical antiretroviral composition may be in the form of a multilayer tablet, such as a bi- or tri-layer tablet, wherein each layer separately contains one or more APIs and pharmaceutically acceptable excipients.

According to an embodiment of the present invention, the pharmaceutical antiretroviral composition comprises lamivudine and nevirapine along with one or more pharmaceutically acceptable excipients to form a layer and festinavir with one or more pharmaceutically acceptable excipients to form another layer, which layers are blended and compressed in a single layer to provide a single unit dosage form.

According to a preferred embodiment, the pharmaceutical antiretroviral composition of the present invention comprises lamivudine and nevirapine along with one or more pharmaceutically acceptable excipients to form a layer and festinavir with one or more pharmaceutically acceptable excipients to form another layer, which layers are blended and compressed to provide a bilayer unit dosage form such as a bilayer tablet.

According to another preferred embodiment, the pharmaceutical antiretroviral composition of the present invention comprises lamivudine along with one or more pharmaceutically acceptable excipients to form a layer and festinavir, nevirapine with one or more pharmaceutically acceptable excipients to form another layer, which layers are blended and compressed to provide a bilayer unit dosage form such as a bilayer tablet.

According to another preferred embodiment, the pharmaceutical antiretroviral composition of the present invention comprises nevirapine along with one or more pharmaceutically acceptable excipients to form layer and lamivudine, festinavir with one or more pharmaceutically acceptable excipients to form another layer, which layers are blended and compressed to provide a bilayer unit dosage form such as a bilayer tablet.

According to another preferred embodiment, the pharmaceutical antiretroviral composition of the present invention comprises nevirapine along with one or more pharmaceutically acceptable excipients to form a layer, lamivudine along with one or more pharmaceutically acceptable excipients to form a layer and festinavir with one or more pharmaceutically acceptable excipients to form another layer, which layers are blended and compressed to provide a trilayer unit dosage form such as a trilayer tablet.

According to another preferred embodiment, the pharmaceutical antiretroviral composition may be administered as a multilayered tablet.

According to one embodiment of the invention, there is provided a process for preparing a pharmaceutical antiretroviral composition which process comprises admixing lamivudine, festinavir and/or nevirapine with one or more pharmaceutically acceptable excipients.

The pharmaceutical antiretroviral composition of the present invention may be prepared through various techniques or processes known in the art which include, but are not limited to, direct compression, wet granulation, dry granulation, melt granulation, melt extrusion, spray drying, solution evaporation or combinations thereof.

It will be acknowledged to a person skilled in the art, that the above-mentioned techniques may be used either singly or in combination with other above-mentioned techniques to provide a single layered, bilayered, tri layered or multilayered unit dosage form.

Suitable pharmaceutically acceptable excipients may be used for formulating the various dosage forms according to the present invention.

According to the present invention, pharmaceutically acceptable carriers, diluents or fillers for use in the pharmaceutical antiretroviral composition of the present invention include, but are not limited to, lactose (for example, spray-dried lactose, α-lactose, β-lactose) white sugar, lactitol, sucrose, saccharose, compressible sugars, sugar confectioners, glucose, calcium carbonate, calcium dihydrogen phosphate dihydrates, calcium phosphate-dibasic, calcium phosphate-tribasic, calcium sulfate, silicified microcrystalline cellulose, cellulose powdered, fructose, kaolin sorbitol, mannitol, dextrates, dextrins, dextrose, maltodextrin, croscarmellose sodium, microcrystalline cellulose, hydroxypropylcellulose, L-hydroxypropylcellulose (low substituted), hydroxypropyl methylcellulose (HPMC), methylcellulose polymers, hydroxyethylcellulose, sodium carboxymethylcellulose, carboxymethylene, carboxymethyl hydroxyethylcellulose and other cellulose derivatives, starches or modified starches (including potato starch, corn starch, maize starch and rice starch) and any mixtures or combinations thereof.

According to the present invention, pharmaceutically acceptable surfactant for use in the pharmaceutical antiretroviral composition of the present invention include, but are not limited to, polysorbates, sodium dodecyl sulfate (sodium lauryl sulfate), lauryl dimethyl amine oxide, docusate sodium, cetyltrimethyl ammonium bromide (CTAB), polyethoxylated alcohols, polyoxyethylenesorbitan, octoxynol, N,N-dimethyldodecylamine-N-oxide, hexadecyltrimethylammonium bromide, polyoxyl 10 lauryl ether, bile salts (sodium deoxycholate, sodium cholate), polyoxyl castor oil, nonylphenolethoxylate, cyclodextrins, lecithin, methylbenzethonium chloride. carboxylates, sulphonates, petroleum sulphonates, alkylbenzenesulphonates, naphthalenesulphonates, olefin sulphonates, alkyl sulphates, sulphates, sulphated natural oils & fats, sulphated esters, sulphatedalkanolamides, alkylphenols (ethoxylated and sulphated), ethoxylated aliphatic alcohol, polyoxyethylene surfactants, carboxylic esters polyethylene glycol esters, anhydrosorbitol ester (and ethoxylated derivatives thereof), glycol esters of fatty acids, carboxylic amides, monoalkanolamine condensates, polyoxyethylene fatty acid amides, quaternary ammonium salts, amines with amide linkages, polyoxyethylene alkyl & alicyclic amines, N,N,N,N tetrakis substituted ethylenediamines, 2-alkyl 1-hydroxyethyl 2-imidazolines, N-coco 3-aminopropionic acid sodium salt, N-tallow-3-iminodipropionate disodium salt, N-carboxymethyl n dimethyl n-9 octadecenyl ammonium hydroxide, n-cocoamidethyl n-hydroxyethylglycine sodium salt, and any mixtures or combinations thereof.

According to the present invention, glidants, anti-adherents and lubricants may also be incorporated in the pharmaceutical antiretroviral composition of the present invention, which include, but are not limited to, stearic acid and pharmaceutically acceptable salts or esters thereof (for example, magnesium stearate, calcium stearate, sodium stearyl fumarate or other metallic stearate), talc, waxes (for example, microcrystalline waxes), glycerides, light mineral oil, PEG, silica acid or a derivative or salt thereof (for example, silicates, silicon dioxide, colloidal silicon dioxide and polymers thereof, crospovidone, magnesium aluminosilicate and or magnesium alumino metasilicate), sucrose ester of fatty acids, hydrogenated vegetable oils (for example, hydrogenated castor oil) mineral oil, stearic acid, colloidal anhydrous silica, sucrose esters of fatty acids, microcrystalline wax, yellow beeswax, white beeswax and any mixtures or combinations thereof.

According to the present invention, suitable binders may also present in the in the pharmaceutical antiretroviral composition of the present invention, which include, but are not limited to, polyvinyl pyrrolidone (also known as povidone), polyethylene glycol(s), acacia, alginic acid, agar, calcium carragenan, cellulose derivatives such as ethyl cellulose, methyl cellulose, hydroxypropyl cellulose, hydroxypropyl methyl cellulose, sodium carboxymethylcellulose, dextrin, gelatin, gum arabic, guar gum, tragacanth, sodium alginate starches, corn starch, pregelatinized starch, microcrystalline celluloses (MCC), silicified MCC, microfine celluloses, lactose, calcium carbonate, calcium sulfate, sugar, mannitol, sorbitol, dextrates, dextrin, maltodextrin, dextrose, dibasic calcium phosphate dihydrate, tribasic calcium phosphate, magnesium carbonate, magnesium oxide, stearic acid, gums, hydroxypropyl methylcelluloses or hypromelloses and any mixtures or combinations thereof.

According to the present invention, suitable disintegrants may also be present in the pharmaceutical antiretroviral composition of the present invention, which include, but are not limited to, hydroxylpropyl cellulose (HPC), low density HPC, carboxymethylcellulose (CMC), sodium CMC, calcium CMC, croscarmellose sodium; starches exemplified under examples of fillers and carboxymethyl starch, hydroxylpropyl starch, modified starch, pregelatinized starch, crystalline cellulose, sodium starch glycolate; alginic acid or a salt thereof, such as sodium alginate or their equivalents and mixtures thereof.

According to the present invention, suitable coloring agents and flavoring agents may also be present in the pharmaceutical antiretroviral composition of the present invention, selected from US FDA approved colors and flavors for oral use.

It would be appreciated by a person skilled in the art, that according to the present invention, the pharmaceutical antiretroviral composition may optionally have one or more coatings, which may be functional or non-functional. Functional coatings include extended-release coatings and non-functional coatings include seal coatings and elegant coatings. Additional excipients such as film forming polymers, solvents, plasticizers, anti-adherents, opacifiers, colorants, pigments, antifoaming agents, and polishing agents can be used in coatings.

Suitable film-forming agents include, but are not limited to, cellulose derivatives, such as, soluble alkyl- or hydroalkyl-cellulose derivatives such as methylcelluloses, hydroxymethyl celluloses, hydroxyethyl celluloses, hydroxypropyl celluloses, hydroxymethylethyl celluloses, hydroxypropyl methylcelluloses, sodium carboxymethyl celluloses, insoluble cellulose derivatives such as ethylcelluloses and the like, dextrins, starches and starch derivatives, polymers based on carbohydrates and derivatives thereof, natural gums such as gum Arabic, xanthans, alginates, polyacrylic acids, polyvinyl alcohols, polyvinyl acetates, polyvinylpyrrolidones, polymethacrylates and derivatives thereof, chitosan and derivatives thereof, shellac and derivatives thereof, waxes, fat substances and any mixtures or combinations thereof.

Suitable enteric coating materials, include, but are not limited to, cellulosic polymers like cellulose acetate phthalates, cellulose acetate trimellitates, hydroxypropyl methylcellulose phthalates, polyvinyl acetate phthalates, methacrylic acid polymers and copolymers and any mixtures or combinations thereof.

Some of the excipients are used as adjuvant to the coating process, including excipients such as plasticizers, opacifiers, antiadhesives, polishing agents, and the like.

Suitable plasticizers include, but are not limited to, castor oil, diacetylated monoglycerides, dibutyl sebacate, diethyl phthalate, glycerin, polyethylene glycols, propylene glycols, triacetin, triethyl citrate, and mixtures thereof.

Suitable opacifier includes, but is not limited to, titanium dioxide.

Suitable anti-adhesive, includes, but is not limited to, talc.

Suitable polishing agents includes, but is not limited to, polyethylene glycols of various molecular weights or mixtures thereof, talc, surfactants (glycerol monostearate and poloxamers), fatty alcohols (stearyl alcohol, cetyl alcohol, lauryl alcohol and myristyl alcohol) and waxes (carnauba wax, candelilla wax and white wax) and mixtures thereof.

Suitable solvents used in the processes of preparing the pharmaceutical antiretroviral composition of the present invention, include, but are not limited to, water, methanol, ethanol, acidified ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethylsulphoxide, N,N-dimethylformamide, tetrahydrofuran, and mixtures thereof.

According to a preferred embodiment, the pharmaceutical antiretroviral composition of the present invention is processed by wet granulation of APIs wherein the diluent, the disintegrant along with the APIs are sifted and dried. Then, binder solution is prepared by first dissolving the binder in purified water. Granulation is carried out by spraying of the binder solution to the above dry mixture of the ingredients, after which the formed granules are dried, sifted through the specified mesh. After unloading, the granules are preferably lubricated.

According to another preferred embodiment, the pharmaceutical antiretroviral composition of the present invention is processed by dry granulation of APIs, wherein the active, diluent, disintegrant are sifted and roll compacted to form granules which are sized by sifting through a specific mesh. After unloading, the granules are preferably lubricated.

Typically, the resulting granules are compressed to provide a single layered tablet or compressed separately to provide a bilayer tablet, a trilayer tablet or a multilayer tablet. The tablets thus obtained via the process may then optionally be sprayed with a coating suspension made of ready colour mix system. Alternatively, after compression into tablets, they can be further seal coated and then sprayed with a film or colour coating suspension or solution. Alternatively, after compression into tablets, they can be further seal coated and then sprayed with a film or colour coating suspension or solution.

In one embodiment of the present invention, the pharmaceutical antiretroviral composition comprises one or more of lamivudine, festinavir and/or nevirapine in “nanosize” (i.e. sub-micron) form. Preferably, one or more of the APIs has a D₅₀ number average particle size of less than 2000 nm, preferably less than 1000 nm more preferably less than 500 nm, such as less than 100 nm, less than 200 nm, less than 300 nm, less than 400 nm, or less than 500 nm. Number average particle size may be measured using suitable particle sizing techniques known in the art such as laser light diffraction.

Nanonization of hydrophobic or poorly water-soluble drugs generally involves the production of drug nanocrystals through either chemical precipitation (bottom-up technology) or disintegration (top-down technology). Different methods may be utilized to reduce the particle size of the hydrophobic or poorly water soluble drugs. [Huabing Chen et al., discusses the various methods to develop nano-formulations in “Nanonization strategies for poorly water-soluble drugs,” Drug Discovery Today, Volume 00, Number 00, March 2010].

Nano-sizing leads to increase in the exposure of surface area of particles leading to an increase in the rate of dissolution. The nanoparticles of the present invention can be obtained by any of the process such as but not limited to milling, precipitation, homogenization, high pressure homogenization, spray-freeze drying, supercritical fluid technology, double emulsion/solvent evaporation, PRINT, thermal condensation, ultrasonication and spray drying.

Accordingly, milling comprises the process of reduction of the size of the particles as such in the solid form by using milling machines such as but not limited to ball mill, jet mill, planetary mill etc. Particle size reduction may also be achieved by dispersing drug particles in a liquid medium in which the drug is poorly soluble followed by applying mechanical means in the presence of grinding media to reduce the particle size of drug to the desired effective average particle size.

Accordingly, the process of precipitation involves the formation of crystalline or semi-crystalline drug nanoparticles by nucleation and the growth of drug crystals. In a typical procedure, drug molecules are first dissolved in an appropriate organic solvent such as acetone, tetrahydrofuran or N-methyl-2-pyrrolidone at a super saturation concentration to allow for the nucleation of drug seeds. Drug nanocrystals are then formed by adding the organic mixture to an antisolvent like water in the presence of stabilizers such surfactants. The choice of solvents and stabilizers and the mixing process are key factors to control the size and stability of the drug nanocrystals.

Accordingly, the process of homogenization involves passing a suspension of crystalline drug and stabilizers through the narrow gap of a homogenizer at high pressure (500-2000 bar). The pressure creates powerful disruptive forces such as cavitation, collision and shearing, which disintegrate coarse particles to nanoparticles.

Accordingly, the process of high pressure homogenization comprises drug presuspension (containing drug in the micrometer range) by subjecting the drug to air jet milling in the presence of an aqueous surfactant solution. The presuspension is then subjected to high-pressure homogenization in which it passes through a very small homogenizer gap of 25 which leads to a high streaming velocity. High-pressure homogenization is based on the principle of cavitations (i.e., the formation, growth, and implosive collapse of vapor bubbles in a liquid).

Accordingly, the process of spray-freeze drying involves the atomization of an aqueous drug solution into a spray chamber filled with a cryogenic liquid (liquid nitrogen) or halocarbon refrigerant such as chlorofluorocarbon or fluorocarbon. The water is removed by sublimation after the liquid droplets solidify.

Accordingly, the process of supercritical fluid technology involves controlled crystallization of drug from dispersion in supercritical fluids, carbon dioxide.

Accordingly, the process of double emulsion/solvent evaporation technique involves preparation of oil/water (o/w) emulsions with subsequent removal of the oil phase through evaporation. The emulsions are prepared by emulsifying the organic phase containing drug, polymer and organic solvent in an aqueous solution containing emulsifier. The organic solvent diffuses out of the polymer phase and into the aqueous phase, and is then evaporated, forming drug-loaded polymeric nanoparticles.

Accordingly, the process of PRINT (Particle replication in non-wetting templates) involves utilization of a low surface energy fluoropolymeric mold that enables high-resolution imprint lithography, to fabricate a variety of organic particles. PRINT can precisely manipulate particle size of drug ranging from 20 nm to more than 100 nm.

Accordingly, the process of thermal condensation involves use of capillary aerosol generator (CAG) to produce high concentration condensation submicron to micron sized aerosols from drug solutions.

Accordingly, the process of ultrasonication involves application of ultrasound during particle synthesis or precipitation, which leads to smaller particles of drug and increased size uniformity.

Accordingly, the process of spray drying involves supplying the feed solution at room temperature and pumping it through the nozzle where it is atomized by the nozzle gas. The atomized solution is then dried by preheated drying gas in a special chamber to remove water moisture from the system, thus forming dry particles of drug.

The pharmaceutical antiretroviral compositions of the present invention comprise the APIs that can be manufactured by any of the types of processes as described above. The processes as describe above, however, do not limit the scope of the invention.

According to a preferred embodiment of the present invention, the nanosize APIs are prepared by wet milling in the presence at least one surface stabilizer, and at least one polymer.

The present invention provides a pharmaceutical antiretroviral composition comprising lamivudine, festinavir and nevirapine for the treatment or prophylaxis of diseases caused by retroviruses, especially acquired immune deficiency syndrome or an HIV infection.

The present invention further provides a pharmaceutical antiretroviral composition comprising lamivudine, festinavir and nevirapine for simultaneous, separate or sequential administration for the treatment or prophylaxis of diseases caused by retroviruses, especially acquired immune deficiency syndrome or an HIV infection.

The following examples are for the purpose of illustration of the invention only and are not intended in any way to limit the scope of the present invention.

### Example 1

### I) Festinavir+Nevirapine Layer

| Sr. No. | Ingredients | Qty/Unit (mg) |
|---|---|---|
| | Dry Mix | |
| 1 | Festinavir | 100.00 |
| 2 | Nevirapine | 200.00 |
| 3 | Microcrystalline cellulose | 100.00 |
| 4 | Sodium starch glycolate | 10.00 |
| 5 | Sunset yellow Lake | 0.40 |
| | Binder | |
| 6 | Corn Starch | 10.00 |
| 7 | Purified water | q.s. |
| | Blending & Lubrication | |
| 8 | Sodium starch glycolate | 10.0 |
| 9 | Microcrystalline cellulose | 64.60 |
| 10 | Magnesium Stearate | 5.00 |

| | |
|---|---|
| Total | 500.0 |

### II) Lamivudine Layer

| Sr. No. | Ingredients | Qty/Unit (mg) |
|---|---|---|
| | Dry Mix | |
| 1 | Lamivudine | 150.00 |
| 2 | Microcrystalline cellulose | 187.00 |
| 3 | Sodium starch glycolate | 10.00 |
| 4 | Colloidal Silicondioxide | 1.0 |
| | Lubrication | |
| 5 | Magnesium Stearate | 2.0 |

| | |
|---|---|
| Total | 350.0 |

Process:

### 1) Festinavir+Lamivudine Layer:

1) Festinavir, Lamivudine, Microcrystalline cellulose, Sunset yellow lake and Sodium starch glycollate were sifted using appropriate sieves.
2) The presifted materials obtained in step (1) were loaded in a mixer granulator and dry mixed.
3) The mixture obtained step (2) was granulated using starch paste to form wet mass,
4) The wet mass obtained in step (3) were sized and dried.
5) The dried granules obtained in step (4) were blended with lubricants.

### II) Nevirapine Layer:

6) Nevirapine, Lactose monohydrate and Hydroxypropylmethylcellulose were sifted using appropriate sieves.
7) The presifted materials obtained in step (6) were loaded in a blender and mixed.
8) The blend obtained in step (7) was compacted by roller compaction and sized to obtain granules.
9) The sized granules obtained in step (8) were blended with lubricants.

### III) Compression

10) The lubricated granules obtained in step (9) were compressed using a bilayer compression machine.

### Example 2

### I) Lamivudine+Nevirapine Layer

| Sr. No. | Ingredients | Qty/Unit (mg) |
|---|---|---|
| | Dry Mix | |
| 1 | Lamivudine | 150.00 |
| 2 | Nevirapine | 200.00 |
| 3 | Microcrystalline cellulose | 50.00 |
| 4 | Sodium starch glycolate | 10.00 |
| 5 | Sunset yellow Lake | 0.40 |
| | Binder | |
| 6 | Corn Starch | 10.00 |
| 7 | Purified water | q.s. |
| | Blending & Lubrication | |
| 8 | Sodium starch glycolate | 10.0 |
| 9 | Microcrystalline cellulose | 64.60 |
| 10 | Magnesium Stearate | 5.00 |

| | |
|---|---|
| Total | 500.0 |

### II) Festinavir Layer

| Sr. No. | Ingredients | Qty/Unit (mg) |
|---|---|---|
| | Dry Mix | |
| 1 | Festinavir | 100.00 |
| 2 | Microcrystalline cellulose | 237.00 |
| 3 | Sodium starch glycolate | 10.00 |
| 4 | Colloidal Silicondioxide | 1.0 |
| | Lubrication | |
| 5 | Magnesium Stearate | 2.0 |

| | |
|---|---|
| Total | 350.0 |

Process:

### 1) Lamivudine+Nevirapine Layer:

1) Festinavir, Nevirapine, Microcrystalline cellulose, Sunset yellow lake and Sodium starch glycollate were sifted using appropriate sieves.
2) The presifted materials obtained in step (1) were loaded in a mixer granulator and dry mixed.
3) The mixture obtained step (2) was granulated using starch paste to form wet mass.
4) The wet mass obtained in step (3) were sized and dried.
5) The dried granules obtained in step (4) were blended with lubricants.

### II) Festinavir Layer:

6) Festinavir, Microcrysalline cellulose, Sodium starch glycollate and Colloidal Anhydrous silica were sifted using appropriate sieves.
7) The presifted materials obtained in step (6) were loaded in a blender and mixed.
8) The blend obtained in step (7) was compacted by roller compaction and sized to obtain granules.
9) The granules obtained were blended with lubricants.

### III) Compression

10) The lubricated granules obtained in step (9) were compressed using a bilayer compression machine.

### Example 3

### I) Fenistavir+Nevirapine Layer

| Sr. No. | Ingredients | Qty/Unit (mg) |
|---|---|---|
| | Dry Mix | |
| 1 | Festinavir | 600.00 |
| 2 | Nevirapine | 400.00 |
| 3 | Microcrystalline cellulose | 100.00 |
| 4 | Sodium starch glycolate | 20.00 |
| 5 | Sunset yellow Lake | 0.60 |
| | Binder | |
| 6 | Corn Starch | 30.00 |
| 7 | Purified water | q.s. |
| | Blending & Lubrication | |
| 8 | Sodium starch glycolate | 20.0 |
| 9 | Microcrystalline cellulose | 116.4 |
| 10 | Magnesium Stearate | 13.00 |

| | |
|---|---|
| Total | 1300.00 |

### II) Lamivudine Layer

| Sr. No. | Ingredients | Qty/Unit (mg) |
|---|---|---|
| | Dry Mix | |
| 1 | Lamivudine | 300.00 |
| 2 | Microcrystalline cellulose | 187.00 |
| 3 | Sodium starch glycolate | 20.00 |
| 4 | Colloidal Silicondioxide | 2.0 |
| | Lubrication | |
| 5 | Magnesium Stearate | 4.0 |

| | |
|---|---|
| Total | 600.0 |

Process:

### 1) Fenistavir+Nevirapine Layer:

1) Festinavir, Nevirapine, Microcrystalline cellulose, Sunset yellow lake and Sodium starch glycollate were sifted using appropriate sieves.
2) The presifted materials obtained in step (1) were loaded in a mixer granulator and dry mixed.
3) The mixture obtained step (2) was granulated using starch paste to form wet mass,
4) The granules obtained in step (3) were sized and dried.
5) The dried granules obtained in step (4) were blended with lubricants.

### II) Lamivudine Layer:

6) Lamivudine, Microcrysalline cellulose, Sodium starch glycollate and Colloidal Anhydrous silica were sifted using appropriate sieves.
7) The presifted materials obtained in step (6) were loaded in a blender and mixed.
8) The blend obtained in step (7) was compacted by roller compaction and sized to obtain granules.
9) The granules obtained were blended with lubricants.

### III) Compression

10) The lubricated granules obtained in step (9) were compressed using a bilayer compression machine.

### Example 4

### I) Fenistavir+Lamivudine Layer

| Sr. No. | Ingredients | Qty/Unit (mg) |
|---|---|---|
| | Dry Mix | |
| 1 | Festinavir | 600.00 |
| 2 | Lamivudine | 300.00 |
| 3 | Microcrystalline cellulose | 116.4 |
| 4 | Sodium starch glycolate | 20.00 |
| 5 | Sunset yellow Lake | 0.60 |
| | Binder | |
| 6 | Corn Starch | 30.00 |
| 7 | Purified water | q.s. |
| | Blending & Lubrication | |
| 8 | Sodium starch glycolate | 20.0 |
| 9 | Magnesium Stearate | 13.00 |

| | |
|---|---|
| Total | 1100.00 |

### II) Nevirapine Layer

| Sr. No. | Ingredients | Qty/Unit (mg) |
|---|---|---|
| | Dry Mix | |
| 1 | Nevirapine | 400.00 |
| 2 | Lactose monohydrate | 126.00 |
| 3 | Hdroxypropylmethylcellulose | 270.00 |
| | Lubrication | |
| 4 | Magnesium Stearate | 4.0 |

| | |
|---|---|
| Total | 600.0 |

Process:

### 1) Fenistavir+Lamivudine Layer:

1) Festinavir, Lamivudine, Microcrystalline cellulose, Sunset yellow lake and Sodium starch glycollate were sifted using appropriate sieves.
2) The presifted materials obtained in step (1) were loaded in a mixer granulator and dry mixed.
3) The mixture obtained step (2) was granulated using starch paste to form wet mass.
4) The granules obtained in step (3) were sized and dried.
5) The dried granules obtained in step (4) were blended with lubricants.

### II) Nevirapine Layer:

6) Nevirapine, Lactose monohydrate and Hydroxypropylmethylcellulose were sifted using appropriate sieves.
7) The presifted materials obtained in step (6) were loaded in a octagonal blender and mixed.
8) The blend obtained in step (7) was compacted by roller compaction and sized to obtain granules.
9) The granules obtained were blended with lubricants.

### III) Compression

10) The lubricated granules obtained were compressed using a bilayer compression machine.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise.

1. A pharmaceutical antiretroviral composition comprising lamivudine, festinavir and nevirapine, or a pharmaceutically acceptable salt, solvate, ester, hydrate, enantiomer, derivative, polymorph, prodrug, complex thereof, and optionally one or more pharmaceutically acceptable excipients.

2. The pharmaceutical antiretroviral composition according to claim 1, in unit dosage form.

3. The pharmaceutical antiretroviral composition according to claim 1, for once or twice daily administration.

4. The pharmaceutical antiretroviral composition according to claim 1, wherein nevirapine is present in the form of an extended release composition.

5. The pharmaceutical antiretroviral composition according to claim 4, wherein nevirapine is formulated with at least one hydrophilic and/or hydrophobic polymer and/or water-swellable polymer.

6. The pharmaceutical antiretroviral composition according to claim 5, wherein nevirapine is coated, blended or complexed with one or more hydrophilic and/or hydrophobic and/or water-swellable polymers.

7. The pharmaceutical antiretroviral composition according to claim 6, wherein the hydrophilic polymer, hydrophobic polymer, water-swellable polymer or mixture thereof, is present in an amount of from about 5% to about 50% by weight of the composition.

8. The pharmaceutical antiretroviral composition according to claim 1 in the form of a solid dosage form.

9. The pharmaceutical antiretroviral composition according to claim 8, in the form of a single layer or multilayer tablet.

10. The pharmaceutical antiretroviral composition according to claim 9, in the form of a bilayer or trilayer tablet.

11. The pharmaceutical antiretroviral composition according to claim 9, comprising:
(i) lamivudine and festinavir and one or more pharmaceutically acceptable excipients to form a first layer; and
(ii) nevirapine in an extended release composition with one or more pharmaceutically acceptable excipients to form a second layer;
which first and second layers are blended and compressed as a single layer to provide a single unit dosage form.

12. The pharmaceutical antiretroviral composition according to claim 9, comprising:
(i) lamivudine and festinavir and one or more pharmaceutically acceptable excipients to form a first layer; and
(ii) nevirapine in an extended release composition with one or more pharmaceutically acceptable excipients to form a second layer;
which first and second layers are blended and compressed to provide a bilayer unit dosage form.

13. The pharmaceutical antiretroviral composition according to claim 9, comprising:
(i) lamivudine with one or more pharmaceutically acceptable excipients to form a first layer; and
(ii) festinavir along with one or more pharmaceutically acceptable excipients to form a second layer; and
(iii) nevirapine in an extended release composition with one or more pharmaceutically acceptable excipients to form a third layer;
which first, second and third layers are blended and compressed to provide a trilayer unit dosage form.

14. The pharmaceutical antiretroviral composition according to claim 1, wherein one or more of the active pharmaceutical ingredients is present in nanosize form, preferably wherein one or more of the active pharmaceutical ingredients has a D₅₀ number average particle size of less than 2000 nm.

15. The process for preparing a pharmaceutical antiretroviral composition according to claim 1, which process comprises the step of admixing lamivudine, festinavir and/or nevirapine, optionally with one or more pharmaceutically acceptable excipients.

16. A pharmaceutical antiretroviral composition comprising lamivudine, festinavir and nevirapine in a kit form.

17. The pharmaceutical composition according to claim 16, comprising a separate unit dosage form of lamivudine and nevirapine or extended release nevirapine and a separate unit dosage form of festinavir.

18. The pharmaceutical composition according to claim 16, comprising a separate unit dosage form of lamivudine and festinavir and a separate unit dosage form of nevirapine or extended release nevirapine.

19. The pharmaceutical composition according to claim 16, comprising a separate unit dosage form of nevirapine or extended release nevirapine and festinavir and a separate unit dosage form of lamivudine.

20. The pharmaceutical composition according to claim 16, comprising a separate unit dosage form of nevirapine or extended release nevirapine, a separate unit dosage form of lamivudine and a separate unit dosage form of festinavir.

21. The pharmaceutical antiretroviral composition according to claim 8, in the form a capsule (filled with powders, pellets, beads, mini-tablets, pills, micro-pellets, small tablet units, MUPS, disintegrating tablets, dispersible tablets, granules, and microspheres, multiparticulates and the like), a soft gelatin capsule, a sachet (filled with powders, pellets, beads, mini-tablets, pills, micro-pellets, small tablet units, MUPS, disintegrating tablets, dispersible tablets, granules, and microspheres, multiparticulates) or in sprinkle form.

22. The pharmaceutical antiretroviral composition according to claim 1, in liquid dosage form.

23. The pharmaceutical composition according to claim 22, in the form of a liquid dispersion, suspension, solution, emulsion, spray, syrup, spot-on formulation, injection preparation, gel, aerosol, ointment, cream, controlled release formulation, lyophilized formulation, delayed release formulation, extended release formulation, pulsatile release formulation, or dual release formulations.

24. The pharmaceutical antiretroviral composition according to claim 1, comprising lamivudine, festinavir and nevirapine as a combined preparation, for simultaneous, separate or sequential administration.

25. The pharmaceutical composition according to claim 24, for use in the treatment or prophylaxis of diseases caused by retroviruses.

26. The pharmaceutical composition according to claim 25, for use in the treatment or prophylaxis of acquired immune deficiency syndrome or an HIV infection.

27. A method for the treatment or prophylaxis of diseases caused by retroviruses, especially acquired immune deficiency syndrome or an HIV infection, which method comprises administering a pharmaceutical antiretroviral composition comprising lamivudine, festinavir and nevirapine to a patient in need thereof.

28. A method utilizing lamivudine, festinavir and nevirapine in the manufacture of a medicament for the treatment or prophylaxis of diseases caused by retroviruses, especially acquired immune deficiency syndrome or an HIV infection.

29. (canceled)

30. (canceled)
